(19) United States
(12) Patent Application Publication
(10) Pub. No.: US 2018/0296739 A1
(43) Pub. Date: Oct. 18, 2018

(54) Suction Device Having Suction Occlusion Capability

(71) Applicant: Eric Cheon, Wilmette, IL (US)

(72) Inventor: Eric Cheon, Wilmette, IL (US)

(21) Appl. No.: 15/955,181

(22) Filed: Apr. 17, 2018

Related U.S. Application Data
(60) Provisional application No. 62/486,949, filed on Apr. 18, 2017.

(51) Int. Cl. A61M 1/00

(52) U.S. Cl. CPC A61M 1/0045; A61M 1/0047; A61M 2209/06; A61M 2205/0205; A61M 2205/42

(57) ABSTRACT

The present disclosure relates generally to a suction device, for example, suitable for use in a medical procedure. The present disclosure relates more particularly to a suction device including a suction tube having a proximal end and a distal end. The suction tube includes a wall surrounding a lumen defining a suction path extending from an orifice at the proximal end to an orifice at the distal end. The suction device also includes an occlusion mechanism attached to the suction tube and including a body having an opening configured to receive suction tubing and inhibit fluid flow through the suction tubing.

### CROSS REFERENCE TO RELATED APPLICATIONS

This application claims the benefit of U.S. Provisional Application No. 62/486,949, filed Apr. 18, 2017, the disclosure of which is hereby incorporated by reference in its entirety.

### BACKGROUND OF THE DISCLOSURE

### 1. Field of the Disclosure

The present disclosure relates generally to a suction device, for example, suitable for use in a medical procedure. The present disclosure relates more particularly to a suction device with an occlusion mechanism.

### 2. Technical Background

Medical suction devices, such as Yankauer suction devices and other aspirators are used to remove substances from a patient's body. For example, a patient may need his/her oropharynx suctioned of debris and liquids to prevent aspiration of these substances into the subglottic trachea or lungs. Existing suction devices are effective for removing substances from the patient's body when needed, but, in many procedures the suction is only required for short periods of time that may be spread out over the course of the procedure. Further, the suction of fluids through the device is typically loud. In particular, when the suction device is not being actively used to remove liquids or debris, the hiss of air passing through the suction device is substantial. Not only is the noise from the suction device distracting, it can also impede communication between medical practitioners, which can cause unnecessary problems and emergency situations.

When the suction device is not being used to remove substances from the patient's body, there is no need for air to pass through the device. However, the machine that creates a vacuum in the suction device is not ordinarily in the immediate reach of the doctor operating on the patient. Accordingly, in order to turn the suction off at the suction machine, the medical practitioner must leave the vicinity of the patient, which is undesirable. Moreover, if use of the suction device is required in an emergency, even a short amount of time needed to move to and from the suction machine can endanger a patient.

The present inventor has recognized that there is a need for a device that can be made quiet when not in use but can quickly resume suction when desired.

### SUMMARY OF THE DISCLOSURE

In one aspect, the present disclosure provides a suction device for a medical procedure, the suction device comprising:
a suction tube having a proximal end and a distal end, the suction tube including a wall surrounding a lumen defining a suction path extending from an orifice at the proximal end to an orifice at the distal end; and
an occlusion mechanism attached to the suction tube and comprising a body including an opening configured to receive suction tubing and inhibit fluid flow through the suction tubing.

In another aspect, the disclosure provides a suction system for a medical procedure, the suction system comprising:
a suction device comprising:
   a suction tube having a proximal end and a distal end, the suction tube including a wall surrounding a lumen defining a suction path extending from an orifice at the proximal end to an orifice at the distal end, and
   an occlusion mechanism attached to the suction tube and comprising a body including an opening; and
suction tubing including a proximal end and a distal end, the distal end of the suction tubing being connected to the proximal end of the suction tube, and a section of the suction tubing between the proximal end and the distal end being disposed in the opening of the occlusion mechanism and held substantially closed by the occlusion mechanism.

In another aspect, the disclosure provides a method of occluding flow through a suction system, the method comprising:
providing a suction system including:
   a suction device including:
      a suction tube having a proximal end and a distal end, and
      an occlusion mechanism attached to the suction tube; and
   suction tubing connected to the proximal end of the suction tube; providing a fluid flow through the suction device and suction tubing; and inserting a section of the suction tubing into the occlusion mechanism so as to
substantially inhibit the fluid flow through the suction device and suction tubing.

Additional aspects of the disclosure will be evident from the disclosure herein.

### BRIEF DESCRIPTION OF THE DRAWINGS

The accompanying drawings are included to provide a further understanding of the methods and devices of the disclosure, and are incorporated in and constitute a part of this specification. The drawings are not necessarily to scale, and sizes of various elements may be distorted for clarity. The drawings illustrate one or more embodiment(s) of the disclosure, and together with the description serve to explain the principles and operation of the disclosure.

FIG. 1 is a schematic side view of a suction device according to an embodiment of the disclosure;

FIG. 2 is a schematic cross-sectional side view of the suction device of FIG. 1;

FIG. 3 is a schematic cross-sectional front view of the suction device of FIG. 1;

FIG. 4 is a schematic side view of the suction device of FIG. 1 with suction tubing inserted into an occlusion mechanism of the suction device;

FIG. 5 is a schematic side view of a suction device according to another embodiment of the disclosure;

FIG. 6 is a schematic side view of a suction device according to another embodiment of the disclosure;

FIG. 7 is a schematic front view of a suction device according to another embodiment of the disclosure;

FIG. 8 is a schematic perspective view of the suction device of FIG. 7;

FIG. 9 is a schematic top view of the suction device of FIG. 7 with suction tubing inserted into an occlusion mechanism of the suction device;

FIG. 10 is a schematic side view of a suction device according to yet another embodiment of the disclosure;

FIG. 11 is a schematic cross-sectional front view of the suction device of FIG. 10

FIG. 12 is a schematic cross-sectional front view of a suction device according to yet another embodiment of the disclosure;

FIG. 13 is a schematic side view of a suction device according to another embodiment of the disclosure;

FIG. 14 is a schematic cross-sectional front view of the suction device of FIG. 13;

FIG. 15 is a schematic side view of a suction device according to another embodiment of the disclosure in a first position;

FIG. 16 is a schematic side view of the suction device of FIG. 15 in a second position;

FIG. 17 is a side view of the suction device of FIG. 1 being held by a person;

FIG. 18 is a schematic side view of a suction device according to another embodiment of the disclosure;

FIG. 19 is a schematic side view of a suction device according to yet another embodiment of the disclosure;

FIG. 20 is a schematic side view of the suction device of FIG. 1 in packaging;

FIG. 21 is a schematic side view of the suction device of FIG. 1 with the packaging of FIG. 20 sheathing a portion of the suction device;

FIG. 22 is a schematic side view of a suction device according to another embodiment of the disclosure;

FIG. 23 is a schematic perspective view of a portion of a suction device according to another embodiment of the disclosure;

FIG. 24 is a schematic perspective view of a portion of a suction device according to yet another embodiment of the disclosure; and

FIG. 25 is a schematic side view of a suction device according to still another embodiment of the disclosure.

### DETAILED DESCRIPTION

As described above, the present inventor has noted that conventional suction devices create a loud and distracting hiss when not in use, and that turning off the suction to the device is inconvenient and possibly hazardous. The present inventor has determined that a suction device in which the suction force and/or suction noise can be reduced easily is advantageous.

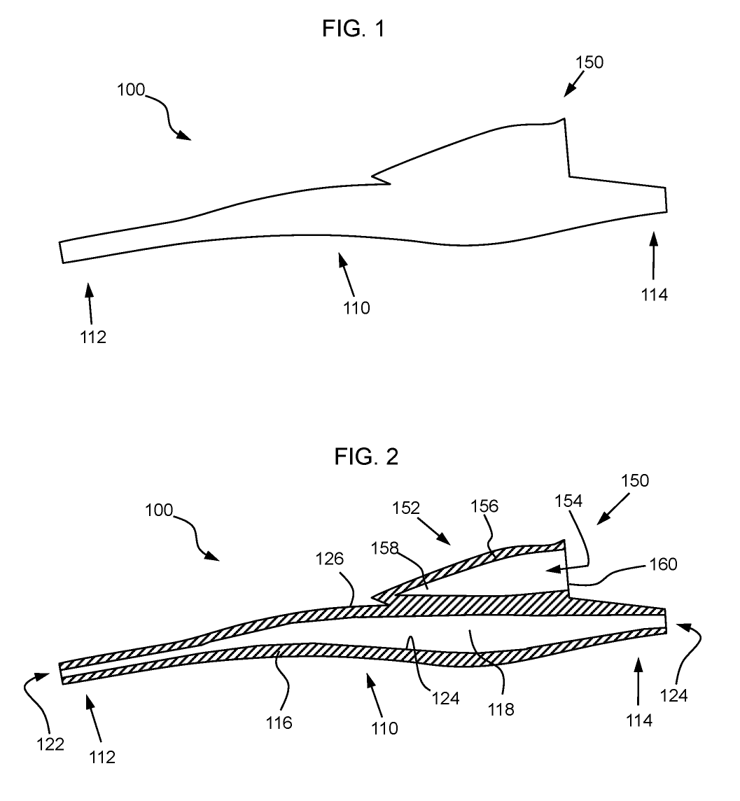

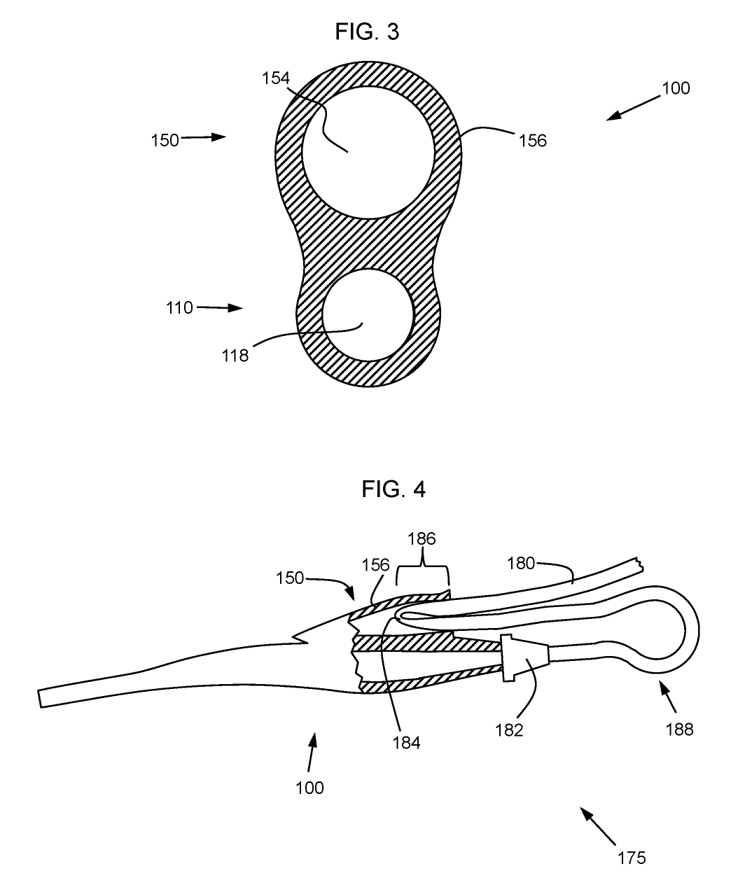

Accordingly, one aspect of the disclosure is a suction device for a medical procedure that includes a suction tube having a proximal end and a distal end. The suction tube includes a wall surrounding a lumen defining a suction path extending from an orifice at the proximal end to an orifice at the distal end. The suction device also includes an occlusion mechanism attached to the suction tube. The occlusion mechanism includes a body having an opening configured to receive suction tubing and inhibit fluid flow through the suction tubing. Such a suction device is schematically shown in FIGS. 1-3. Suction device 100 includes a suction tube 110 and an occlusion mechanism 150 that is configured to receive suction tubing and hold the suction tubing in an occluded position in order to inhibit fluid flow therethrough, as explained in more detail below. Suction tube 110 has an elongate shape with a distal end 112 and a proximal end 114. A wall 116 of suction tube 110 has a closed cross section so as to define a lumen 118 that passes through the suction tube, as shown in the cross sectional views of FIGS. 2 and 3. Lumen 118 extends across suction tube 110 from an inlet orifice 122 at distal end 112 to an outlet orifice 124 at proximal end 114. The lumen 118 forms a suction path for removing substances from a patient when coupled to a suction source.

The term “distal,” as used herein, refers to the direction that is closest to the patient while the term “proximal,” as used herein, refers to the direction closest to the clinician and the suction source. In normal operation, fluid flowing through the suction tube moves from the distal end to the proximal end.

While both the inner and outer surfaces of wall 116 of suction tube 110 are round and circular in shape, in other embodiments, these surfaces have other shapes. For example, in some embodiments, the inner surface of the suction tube is in the shape of a polygon, such as a hexagon. In other embodiments, the outer surface of the suction tube is in the shape of a polygon. Still in other embodiments, both the inner and outer surfaces are polygonal. Still in other embodiments, the suction tube has a complex shape including both rounded portions and flat portions.

The occlusion mechanism 150 includes a body 152 having an opening 154 that is formed as a hollow chamber surrounded by a retaining wall 156. In operation, the opening 154 of body 152 provides a space to insert a section of tubing 180, as shown in system 175 depicted in FIG. 4. Further, retaining wall 156 of body 152 holds the suction tubing 180 in a folded and kinked position to occlude the tubing. In other embodiments, as described in more detail below, the occlusion mechanism holds the tubing in an occluded position without a kinked fold in the tubing.

As described herein, the suction tube is a part of the suction device, while suction tubing is separate from and attached to the suction tube. For example, in system 175, suction tubing 180 is attached to the proximal end 114 of suction tube 110 via an adapter 182. In certain embodiments, the suction tubing extends from the suction device to a suction source, such as an anesthesia machine.

Embodiments of the suction device include a variety of different sizes. The length of the suction device, the outer diameter of the suction tube, the inner diameter of the suction tube, the wall thickness, and the size of the occlusion mechanism each can be made in a range of sizes, as will be appreciated by those of ordinary skill in the art. In some embodiments the length of the suction tube is in a range from 4 inches to 2 feet, e.g., from 6 inches to 12 inches. In certain embodiments the cross sectional width, e.g., diameter, of the inlet orifice and the outlet orifice are in a range from 1/10 inch to 1 inch, e.g., from ⅛ inch to ¾ inch. In some embodiments the lumen widens between the inlet orifice and the outlet orifice. In certain embodiments the largest cross sectional width of the lumen is in a range from ¼ inch to 1½ inches, e.g., ⅓ inch to 1 inch. In some embodiments, the opening of the occlusion mechanism has a cross sectional width in a range of ⅓ inch to 1½ inches. In certain embodiments, the wall thickness of the suction tube is in a range of 1/30 inch to ¼ inch.

In certain embodiments as otherwise described herein, the occlusion mechanism is disposed on an outer surface of the wall of the suction tube. For example, as shown in FIG. 2, wall 116 of suction tube 110 includes an inner surface 124 that surrounds lumen 118 and an outer surface 126. Occlusion mechanism 150 is disposed on the outer surface 126 of wall 116. Indeed, occlusion mechanism 150 abuts suction tube 110 and runs along the outer surface 126 of wall 116. As a result, retaining wall 156 is coextensive with suction tube wall 116 in the area where occlusion mechanism 150 and suction tube 110 are adjacent. In other embodiments, the occlusion mechanism is in another location. For example, in some embodiments the occlusion mechanism is aligned with the suction path provided by the lumen. In other embodiments, the occlusion mechanism is more remote, as in suction device 1900, described in more detail below.

In certain embodiments as otherwise described herein, the occlusion mechanism includes an open end providing access to the opening and a closed end opposite the open end. For example, occlusion mechanism 150 in suction device 100 includes an open end 160 facing the proximal side 114 of the suction device and a closed end 158. Open end 160 provides access to opening 154 so that tubing can be inserted therein. Further retaining wall 156 tapers toward closed end 158. The tapering of retaining wall 156 promotes kinking of any suction tubing inserted therein as the suction tubing is pushed toward the closed end 158 of occlusion mechanism 150. In other embodiments, the closed end is formed by an end wall of the occlusion mechanism, without any tapering of the retaining wall or opening.

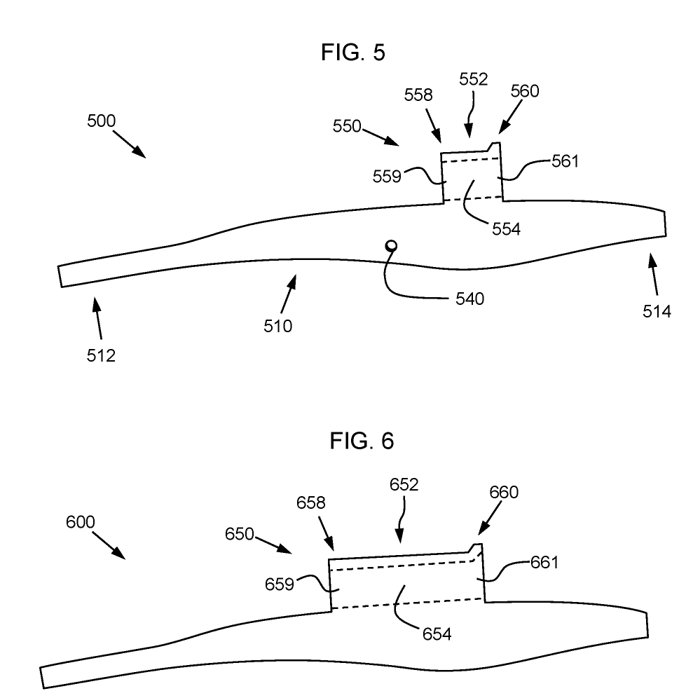

In certain embodiments as otherwise described herein, the opening extends through the body of the occlusion mechanism. The phrase “extends through,” as used herein, refers to the opening going all the way through the body of the occlusion mechanism, such that the opening is accessible from either end of the occlusion mechanism. For example, the body 552 of occlusion mechanism 550 of suction device 500, shown in FIG. 5, is in the form of a ring that has an orifice 559 at its distal end 558 and an orifice 561 at its proximal end 560. In operation, tubing can be inserted into opening 554 such that a kink is held within the ring-shaped body 552. Alternatively, the tubing may be inserted all the way through body 552 so that the kinked end of the tubing extends beyond the orifice 559 at the distal end 558 of the occlusion mechanism 550. Likewise, the tubing may be inserted from the distal end toward the proximal end.

Occlusion mechanism 650 of suction device 600 also includes an opening that extends through the body 652. In particular, body 652 of occlusion mechanism 650 is in the shape of a tube, where the opening 654 extends from an orifice 659 at the distal end 658 thereof to an orifice 661 at the proximal end 660. As described above with respect to suction device 500, suction tubing may be inserted into body 652 so that a kink is held within the occlusion mechanism 650, or it may be inserted all the way through the occlusion mechanism 650 with the kinked portion disposed beyond the distal end 658.

In certain embodiments, the orifice at the proximal end of the opening of the occlusion mechanism is tapered so as to promote insertion of tubing in to the occlusion mechanism. For example, opening 654 tapers inward slightly from orifice 661, which guides insertion of tubing into the opening, as will be appreciated by those of ordinary skill in the art.

In certain embodiments as otherwise described herein, the body of the occlusion mechanism is a closed loop that surrounds the opening. For example, the ring-shaped body 552 of occlusion mechanism 550 and the tube-shaped body 652 of occlusion mechanism 650 are closed loops that surround the opening. In particular, the retaining wall of each body fully extends around the respective opening. Likewise, retaining wall 156 of occlusion mechanism 150 of suction device 100 is formed as a closed loop that extends around and surrounds opening 154, as shown in FIG. 3.

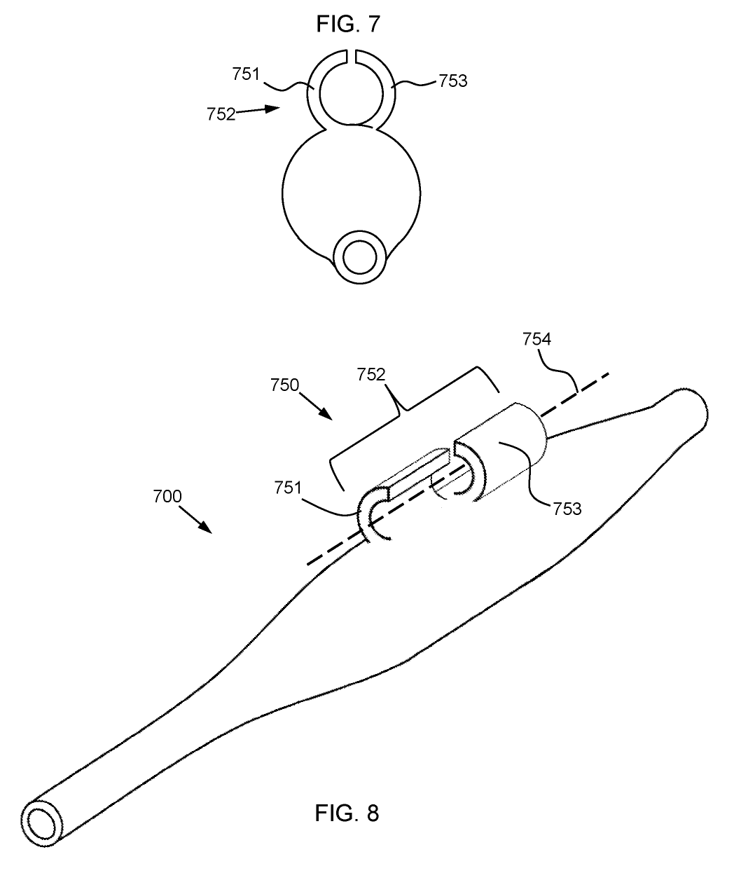

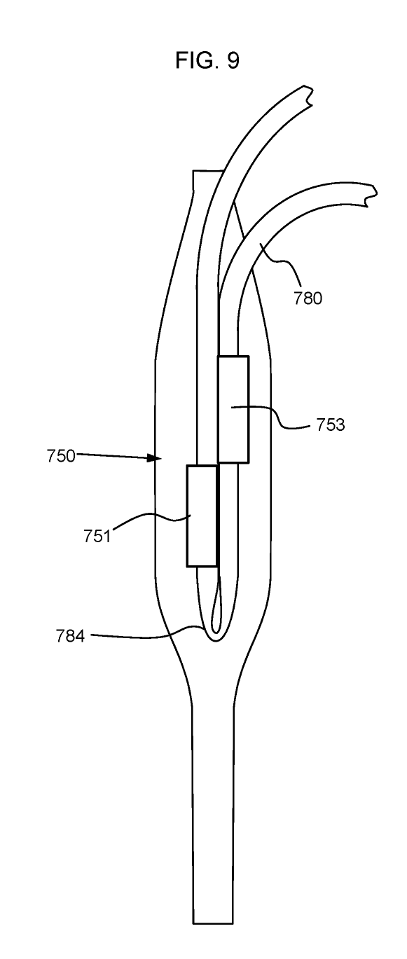

In other embodiments, the body of the occlusion mechanism is formed by distinct pieces that form an opening therethrough without extending in a closed loop. For example, occlusion mechanism 750 of suction device 700, shown in FIGS. 7-9, includes a body 752 that is formed by two wall segments 751, 753. Each of the segments 751, 753 has the shape of a semicircular tab that face opposing directions. Accordingly, an opening 754, shown by the dashed line in FIG. 8, is provided through the two segments 751, 753 for holding occluded tubing therein. For example, as shown in FIG. 9, tubing 780 may be inserted through the opening between segments 751 and 753 so as to hold an occluded kinked section 784 in occlusion mechanism 750.

In certain embodiments as otherwise described herein, the suction device is formed as a single integrally formed piece. For example, in some embodiments, the suction device is formed in a single mold. In certain embodiments, the suction device is made of a flexible material. In other embodiments, the suction device is made of a stiff material, such as a hard plastic. Suitable materials for embodiments of the suction device formed of a single material include medical grade polymer, silicone, polyvinyl chlorides, urethanes, rubbers (including synthetic rubbers) and metals. Other materials may also be used, as will be appreciated by those of ordinary skill in the art.

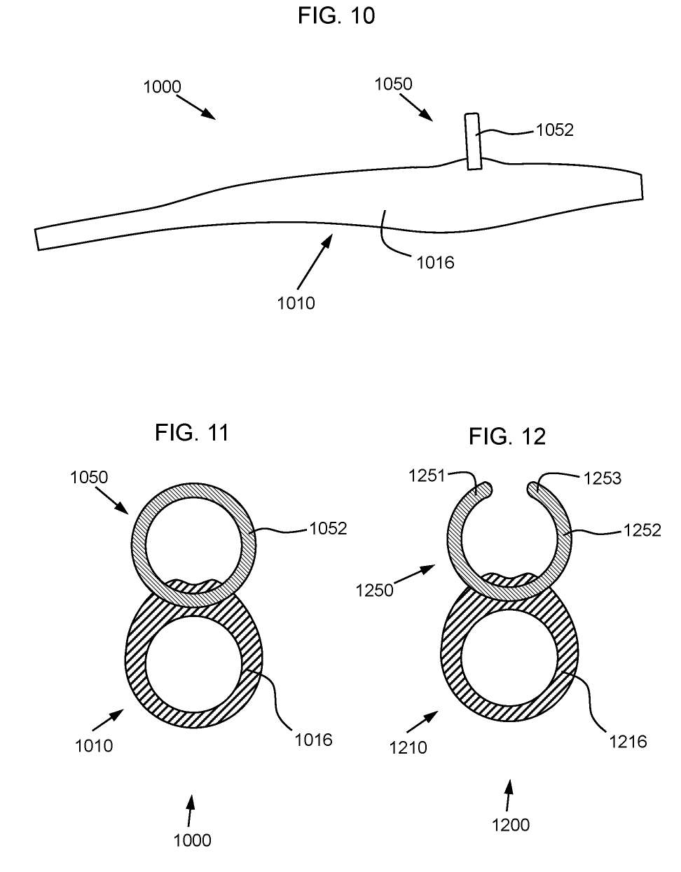

In certain embodiments as otherwise described herein, the suction device is formed from a plurality of materials. For example, such a suction device is shown in FIGS. 10 and 11. Suction device 1000 includes a suction tube 1010 formed from a first material and an occlusion mechanism 1050 formed from a second material. In particular, suction tube 1010 is formed from a polymer and the body 1052 of occlusion mechanism 1050 is formed as a metal ring that is partially embedded in the wall 1016 of suction tube 1010. Suction device 1200 shown in FIG. 12 includes a similar configuration, with occlusion mechanism 1250 partially embedded in suction tube 1210. Notably, the body 1252 of occlusion mechanism 1250 is formed with slight opening between two segments 1251, 1253, rather than as a closed loop.

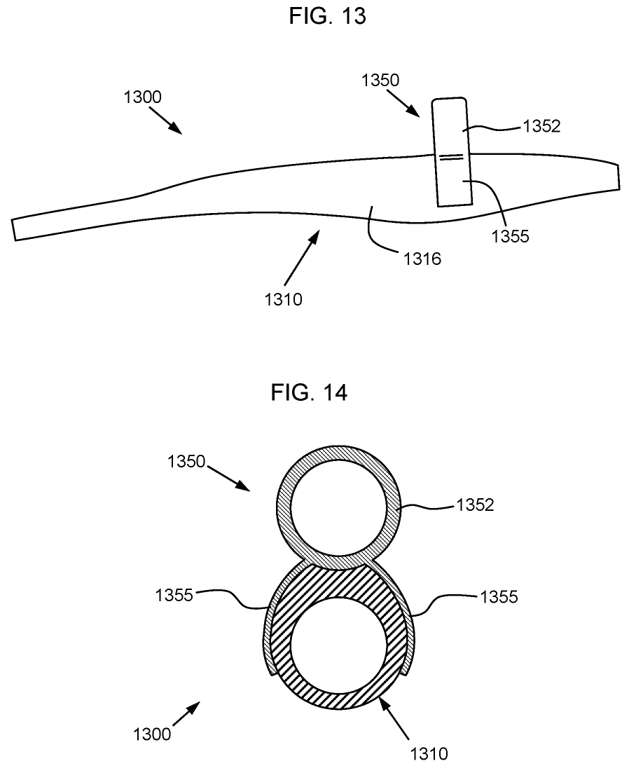

In certain embodiments as otherwise described herein, the occlusion mechanism is detachable from the suction tube. For example, suction device 1300, shown in FIG. 13, includes an occlusion mechanism 1350 that is detachable from suction tube 1310. Specifically, two arms 1355 extend from body 1352 so that occlusion mechanism 1350 can be removably attached to suction tube 1310. The arms 1355 are flexible so that they can bend outward as the suction tube is inserted therebetween. The arms will then return to their original position, partially wrapping around suction tube 1310 in order to provide a secure connection between occlusion mechanism 1350 and suction tube 1310. In other embodiments the occlusion mechanism can be attached to the suction tube in a variety of ways, as will be appreciated by those of ordinary skill in the art.

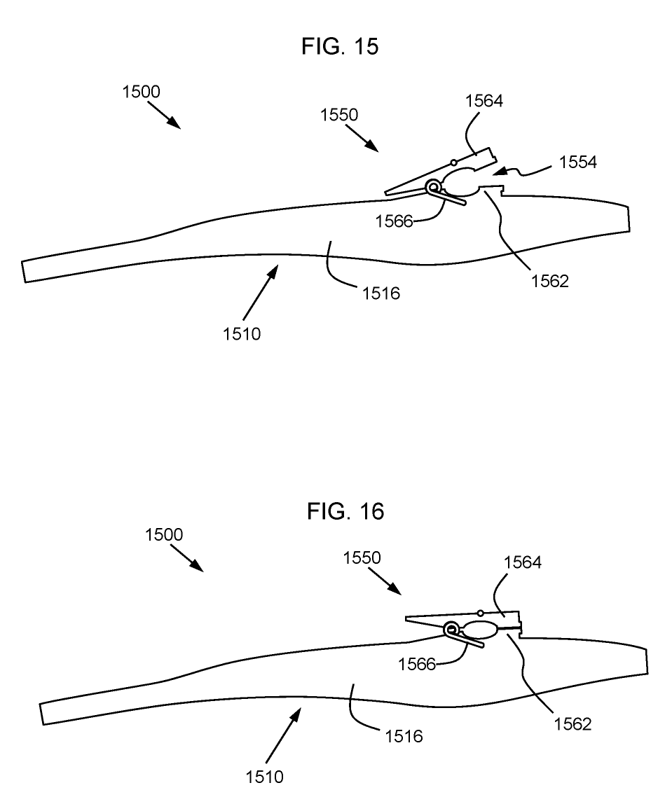

In certain embodiments as otherwise described herein, the opening is closeable. In particular, in some embodiments the opening is configured to close so as to occlude the tubing disposed in the occlusion mechanism. For example, suction device 1500 shown in FIGS. 15 and 16 includes an occlusion mechanism 1550 in the form of a spring-based clamp. The body of the occlusion mechanism includes a lower member 1562 integrally formed with the wall 1516 of suction tube 1510 and an upper member 1564 that is coupled to the lower member using a spring 1566. The opening 1554 of the occlusion mechanism 1550 is closeable by the force of spring 1566 but may be opened by applying a downward force at the distal end of upper member 1564. In use, the clamp can be opened so that tubing may be inserted into the opening 1554. The force provided by spring 1566 will then close opening 1554 in order to squeeze the tubing shut and occlude the tubing.

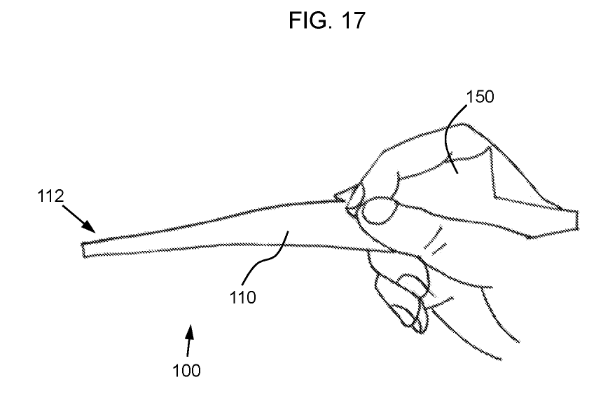

In certain embodiments as otherwise described herein, the occlusion mechanism and an adjacent portion of the suction tube form a handle for manipulating the suction device. For example, the occlusion mechanism 150 and suction tube 110 of suction device 100 are arranged so as to form a handle to carefully control the location of the distal end 112 of the suction tube. An illustration of the suction device 100 being held in a manner similar to a pen using the handle formed by the occlusion mechanism 150 and suction tube 110 is shown in FIG. 17.

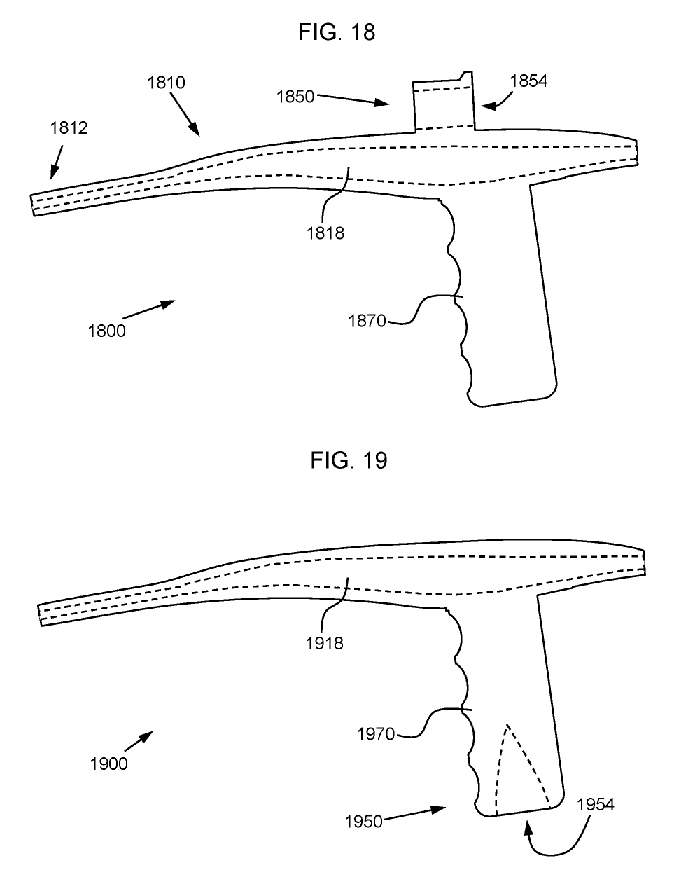

In other embodiments, the suction device includes a handle that is attached to the suction tube. For example, in suction device 1800, shown in FIG. 18, a handle 1870 extends down from the suction tube 1810 in an area near the proximal end of the suction device. The handle 1870 includes grooves for a user's fingers and the shape of the suction device, which is similar to a gun, allows for careful and accurate manipulation of the distal end 1812 of the suction device. Similar to other embodiments described above, the occlusion mechanism 1850 is disposed above suction tube 1810. In other embodiments, the occlusion mechanism is included in the handle. For example, opening 1954 of occlusion mechanism 1950 in suction device 1900, shown in FIG. 19, is formed in the lower end of handle 1970.

In certain embodiments as otherwise described herein, a depth direction of the opening of the occlusion mechanism and the direction of the suction path are substantially parallel. The phrase “substantially parallel,” as used herein, refers to lines that are within 20 degrees of one another. Further, the direction of the suction path is defined as a line that extends from the orifice at the distal end to the orifice at the proximal end of the suction device, where the path through the suction tube is relatively linear and does not curve more than 35 degrees. For example, in suction device 1800, the depth direction of opening 1854 of occlusion mechanism 1800 and the direction of the suction path through lumen 1818 are substantially parallel. In contrast, the depth direction of opening 1954 of occlusion mechanism 1950 is substantially perpendicular to the direction of the suction path through lumen 1918.

In certain embodiments as otherwise described herein, the depth direction of the opening of the occlusion mechanism is coplanar with the suction path through the suction tube. For example, in both suction device 1800 and device 1900 the suction path provided by the respective lumen in the suction tube and the depth direction of the opening of the occlusion mechanism lie in the same plane. In other embodiments, the line along the suction path and the line defined by the depth direction of the opening form skew lines. For example, an embodiment similar to that of suction device 1000, but where the ring-shaped body is rotated so that the tubing is inserted from the side, includes a suction path direction and an opening depth direction that form skew lines.

In certain embodiments as otherwise described herein, a proximal end of the occlusion mechanism is spaced from the proximal end of the suction tube. For example, the proximal end 560 of occlusion mechanism 550 in suction device 500 is spaced back from the proximal end 514 of suction tube 510. This positioning of occlusion mechanism 550 provides a shelf over the proximal end 514 of the suction tube 510, which allows tubing to be placed on the suction tube for insertion into the occlusion mechanism.

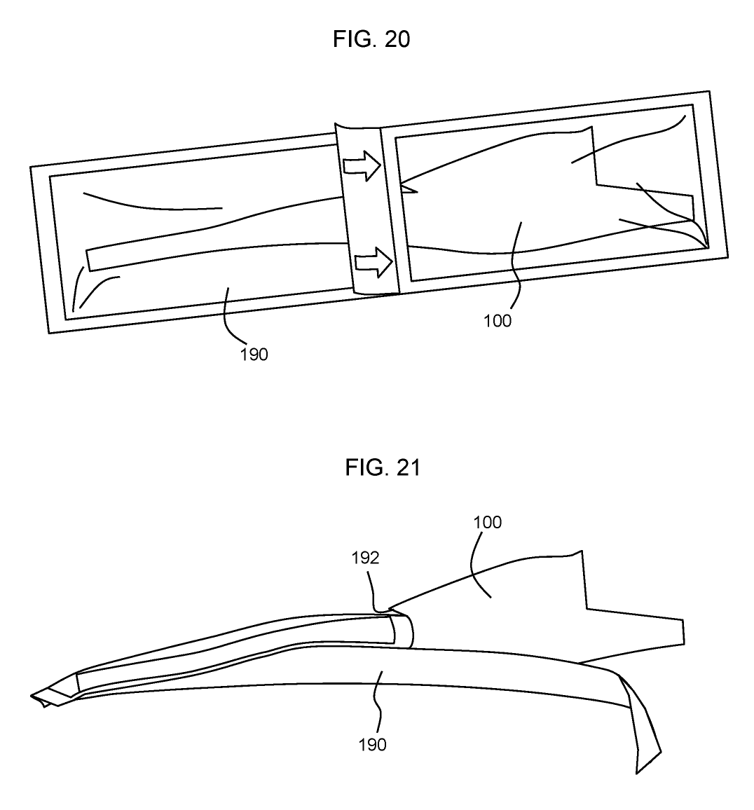

In certain embodiments as otherwise described herein, the suction device further includes a notch in an outer surface of the wall of the suction tube. The notch is configured to securely receive an edge of a product wrapper therein. For example, suction device 100 is shown in FIG. 20 disposed within a product wrapper 190. The wrapper is sealed on all sides to keep the device sterile until use. Upon use, the opening tab of the wrapper is pulled in the direction shown by the arrows to break the seal and access the suction device 100. Once the wrapper has been opened, the distal end of suction device 100 can be re-sheathed within the wrapper to provide limited protection of the device against soiling. To hold the wrapper in place, an edge of the wrapper may be inserted into notch 192, as shown in FIG. 21, while the suction device is partially sheathed in the wrapper.

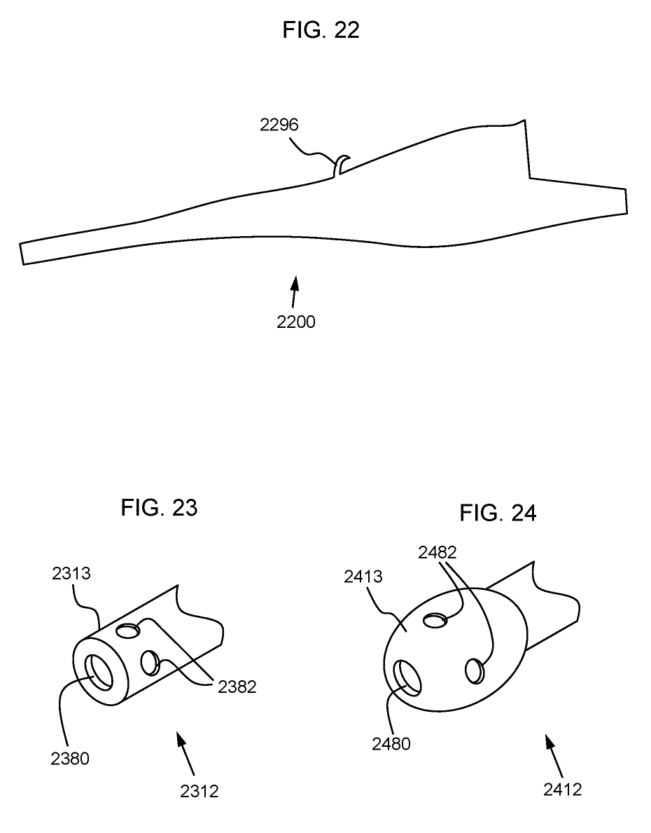

In certain embodiments as otherwise described herein, the suction device includes a protrusion for holding a wrapper over the suction device. For example, the protrusion may be in the form of a pin, a hook or a stud that passes through (or forms) a small hole in the wrapper to secure the wrapper on the suction device. For example, suction device 2200, shown in FIG. 22 includes protrusion 2296 in the form of a hook for holding a wrapper that is sheathed over a portion of the suction device.

In certain embodiments as otherwise described herein, the tip at the distal end of the suction tube includes multiple perforations for receiving substances from a patient's body. For example, FIG. 23 shows the distal end 2312 of a suction tube according to an embodiment of the disclosure, which includes a tip 2313 having a plurality of perforations therein. The perforations include an end perforation 2380 at the end of the suction tube and side perforations 2382 around the tip 2313. In certain embodiments as otherwise described herein, the distal end of the suction tube includes a bulbous tip. For example, FIG. 24 shows the distal end 2412 of a suction tube that includes a bulbous tip 2413. The bulbous tip also includes a plurality of perforations 2480, 2482.

In certain embodiments as otherwise described herein, the wall of the suction tube includes an aperture between the proximal end and the distal end, wherein the aperture is configured to control suction through the lumen. For example, suction device 500 includes an aperture 540 that can be covered to increase the flow through the distal end 512 of suction tube 510 or uncovered to decrease the flow through the distal end. Closing of aperture 540 can be conveniently carried out by the medical clinician using a finger to cover the aperture. In some embodiments, the suction device includes more than one aperture for controlling flow through the distal end of the suction tube. For example, in some embodiments, the suction tube includes a larger aperture and a smaller aperture, where either or both apertures can be covered so as to achieve four different flow volumes.

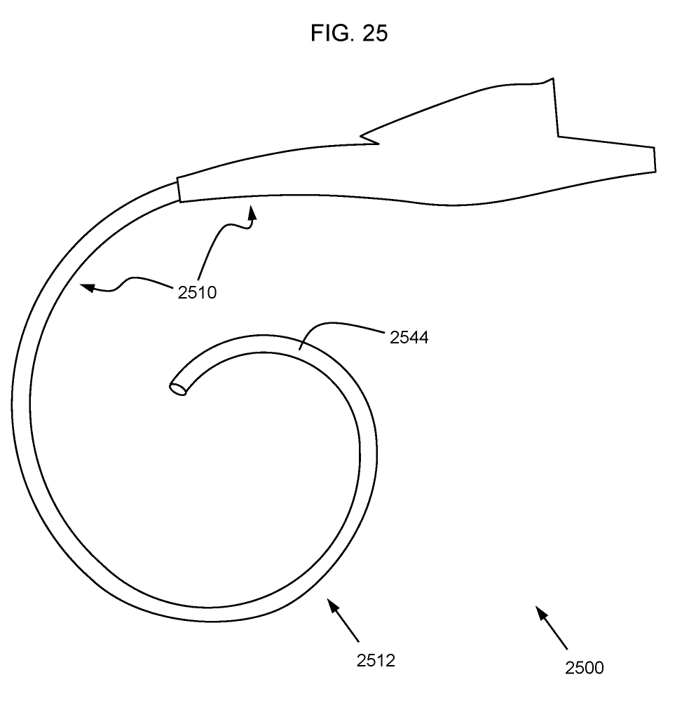

In certain embodiments as otherwise described herein, the distal end of the suction tube is flexible. For example, such a suction device is shown in FIG. 25. Suction device 2500 includes a long flexible portion 2544 of suction tube 2510 located at the distal end 2512 of the tube. The flexible portion 2544 can be inserted into a patient's body to provide steady suction in a certain area.

In certain embodiments as otherwise described herein, at least a portion of the suction tube includes an antimicrobial coating thereon. Such a coating can reduce the microbial load, and colonization on the suction tube. Suitable antimicrobial materials include, but are not limited to, silver nanoparticles, copper nanoparticles, antibiotic-impregnated cyclodextrin, quarternary ammonium/imidazolium salt, or polyethyleneimine. Other coatings are also possible, as will be appreciated by those of ordinary skill in the art.

In another aspect, the disclosure provides a suction system for a medical procedure. The system includes a suction device according to any of the above embodiments, and suction tubing including a proximal end and a distal end. The distal end of the suction tubing is connected to the proximal end of the suction tube and a section of the suction tubing between the proximal end and distal end is secured in the opening of the occlusion mechanism and held substantially closed by the occlusion mechanism. For example, such a system is shown in FIG. 4. System 175 includes suction tubing 180 connected to the proximal end 114 of suction tube 110. A section 186 of the suction tubing is secured in the occlusion mechanism 150 and flow through the tubing is substantially inhibited by the occlusion mechanism and the sound of fluid flow through the tubing is muted. The term “occlude,” as used herein, refers to a reduction in volume flow of at least 90% compared to a volume flow where the tubing is open and flow in unimpeded. The retaining wall 156 provides a force against the tubing 180 that holds the section of tubing securely in the occlusion mechanism.

In certain embodiments as otherwise described herein, the section of suction tubing is folded to form a kink that substantially closes the suction tubing, and wherein the folded section of suction tubing is held in the opening in the occlusion mechanism. For example, tubing 180 in system 175 is folded so as to form a kink 184 within section 186 of the tubing. The kinked section is disposed in the opening 154 so that the retaining wall 156 holds the kinked section therein in the closed position.

In certain embodiments as otherwise described herein, the occlusion mechanism is disposed on an outer surface of the wall of the suction tube, and wherein a portion of the suction tubing between the distal end and the section disposed in the opening of the occlusion mechanism forms a loop. For example, suction tubing 180 in system 175 provides a loop 188 between the kink 184 and the adapter 182 that is connected to the suction tube 110. This loop can be used to hang the suction device while not in use. For example, the suction device may be hung from a variety of structures, including but not limited to an anesthesia circuit tree, the side rails of an operating room table, an anesthesia machine, or an IV stand.

In certain embodiments as otherwise described herein, the system further includes a suction pump coupled to the proximal end of the suction tubing. In some embodiments, the suction pump is formed by an vacuum pump, a fan or an impeller. In some embodiments the suction pump is part of an anesthesia machine.

In another aspect, the present disclosure provides a method of occluding flow through a suction system. The method includes providing a suction system including a suction device according to the disclosure and suction tubing connected to the proximal end of the suction tube. The method includes providing a fluid flow through the suction tubing and inserting a section of the suction tubing into the occlusion mechanism so as to substantially inhibit the fluid flow through the suction device and suction tubing. The phrase “fluid flow,” as used herein, includes both liquids and gases, and also includes debris or particulates carried in a flow by the respective fluid. For example, during a medical procedure, after a clinician uses the suction device, she inserts the suction tubing into the occlusion mechanism so as to occlude the flow therethrough and mute the sound of fluid flowing through the suction path.

In certain embodiments as otherwise described herein, the method includes pushing the section of suction tubing into the occlusion mechanism into the opening so as to form a kink in the section of suction tubing that substantially inhibits the fluid flow. For example, as suction tubing 180 of system 175 is pushed into occlusion mechanism 150, the retaining wall 156 will press against the tubing until a kink is formed in the tubing that occludes the flow.

In certain embodiments as otherwise described herein, the method includes folding the section of suction tubing so as to form a kink in the section of suction tubing that substantially inhibits the fluid flow prior to inserting the section into the occlusion mechanism. For example, the clinician can immediately occlude the suction tubing by folding the tubing so as to form a kink therein. The tubing can then be inserted into the occlusion mechanism in order to hold the section of tubing in the folded position and retain the kinked portion.

In certain embodiments as otherwise described herein, the occlusion mechanism extends outward from a wall of the suction tube and a portion of the suction tubing between the distal end and the section disposed in the occlusion mechanism forms a loop. The method further includes hanging the suction device on a support structure using the loop of the suction tubing. For example, once the section of tubing is inserted into the occlusion mechanism, a loop is formed between the occlusion mechanism and the connection between the tubing and the proximal end of the suction tube. This loop can be used to hang the suction device and attached tubing onto a support structure, examples of which are provided above.

In certain embodiments as otherwise described herein, the method further includes removing the section of suction tubing from the occlusion mechanism so as to restart the fluid flow through the suction device and suction tubing. For example, to resume suction through the device, the clinician can quickly pull the suction tubing from the occlusion mechanism so as to reinstate the flow.

It will be apparent to those skilled in the art that various modifications and variations can be made to the processes and devices described here without departing from the scope of the disclosure. Thus, it is intended that the present disclosure cover such modifications and variations of this invention provided they come within the scope of the appended claims and their equivalents.

What is claimed is:

1. A suction device for a medical procedure, the suction device comprising:
a suction tube having a proximal end and a distal end, the suction tube including a wall surrounding a lumen defining a suction path extending from an orifice at the proximal end to an orifice at the distal end; and
an occlusion mechanism attached to the suction tube and comprising a body including an opening configured to receive suction tubing and inhibit fluid flow through the suction tubing.

2. The suction device according to claim 1, wherein the occlusion mechanism is disposed on an outer surface of the wall of the suction tube.

3. The suction device according to claim 1, wherein the occlusion mechanism includes an open end providing access to the opening and a closed end opposite the open end.

4. The suction device according to claim 3, wherein the occlusion mechanism and an adjacent portion of the suction tube form a handle for manipulating the suction device.

5. The suction device according to claim 1, wherein the opening extends through the body of the occlusion mechanism.

6. The suction device according to claim 1, wherein the body of the occlusion mechanism is a closed loop that surrounds the opening.

7. The suction device according to claim 1, wherein a proximal end of the occlusion mechanism is spaced from the proximal end of the suction tube.

8. The suction device according to claim 1, wherein a depth direction of the opening of the occlusion mechanism is coplanar with the suction path through the suction tube.

9. The suction device according to claim 1, further comprising a notch in an outer surface of the wall of the suction tube, the notch being configured to securely receive an edge of a product wrapper therein.

10. The suction device according to claim 1, wherein the distal end of the suction tube includes a plurality of perforations.

11. The suction device according to claim 1, wherein the wall of the suction tube includes an aperture between the proximal end and the distal end, wherein the aperture is configured to control suction through the lumen.

12. A suction system for a medical procedure, the suction system comprising:
a suction device comprising:
a suction tube having a proximal end and a distal end, the suction tube including a wall surrounding a lumen defining a suction path extending from an orifice at the proximal end to an orifice at the distal end, and
an occlusion mechanism attached to the suction tube and comprising a body including an opening; and
suction tubing including a proximal end and a distal end, the distal end of the suction tubing being connected to the proximal end of the suction tube, and a section of the suction tubing between the proximal end and the distal end being secured in the opening of the occlusion mechanism and held substantially closed by the occlusion mechanism.

13. The suction system according to claim 12, wherein the section of suction tubing is folded to form a kink that substantially closes the suction tubing, and wherein the folded section of suction tubing is inserted into the opening in the occlusion mechanism.

14. The suction system according to claim 12, wherein the occlusion mechanism is disposed on an outer surface of the wall of the suction tube, and wherein a portion of the suction tubing between the distal end and the section disposed in the opening of the occlusion mechanism forms a loop.

15. The suction system according to claim 12, further comprising a suction pump coupled to the proximal end of the suction tubing.

16. A method of occluding flow through a suction system, the method comprising:
providing a suction system including:
a suction device including:
a suction tube having a proximal end and a distal end, and
an occlusion mechanism attached to the suction tube, and
suction tubing connected to the proximal end of the suction tube;
providing a fluid flow through the suction device and suction tubing; and
inserting a section of the suction tubing into the occlusion mechanism so as to substantially inhibit the fluid flow through the suction device and suction tubing.

17. The method according to claim 16, further comprising pushing the section of suction tubing into the occlusion mechanism into the opening so as to form a kink in the section of suction tubing that substantially inhibits the fluid flow.

18. The method according to claim 16, further comprising folding the section of suction tubing so as to form a kink in the section of suction tubing that substantially inhibits the fluid flow prior to inserting the section into the occlusion mechanism.

19. The method according to claim 16, wherein the occlusion mechanism extends outward from a wall of the suction tube, and wherein a portion of the suction tubing between the distal end and the section disposed in the occlusion mechanism forms a loop,
the method further comprising hanging the suction device on a support structure using the loop of suction tubing.

20. The method according to claim 16, further comprising removing the section of suction tubing from the occlusion mechanism so as to restart the fluid flow through the suction device and suction tubing.
